(19) United States
(12) Patent Application Publication
(10) Pub. No.: US 2025/0249273 A1
(43) Pub. Date: Aug. 7, 2025

(54) Localizing, Imaging, and Heating Magnetic Nanoparticles Using Magnetic Nanoparticle Magnetization Controlled Through Electron Paramagnetic Resonance and Ferromagnetic Resonance

(71) Applicant: Mary Hitchcock Memorial Hospital, for itself and on behalf of Dartmouth-Hitchcock Clinic, Lebanon, NH (US)

(72) Inventor: John B. Weaver, Hanover, NH (US)

(21) Appl. No.: 19/085,747

(22) Filed: Mar. 20, 2025

Related U.S. Application Data
(63) Continuation-in-part of application No. 18/116,742, filed on Mar. 2, 2023, now abandoned.
(60) Provisional application No. 63/315,626, filed on Mar. 2, 2022.

(51) Int. Cl. A61N 2/02; A61N 2/00; G01K 7/38; G01R 33/389; G01R 33/60

(52) U.S. Cl. CPC A61N 2/02; A61N 2/004; G01R 33/389; G01K 7/38; G01K 2211/00; G01K 2213/00; G01R 33/60

(57) ABSTRACT

An MNP machine provides a bias field consisting of a low frequency alternating magnetic field and possibly a static magnetic field to a volume in possibly different directions; RF drive coils driven at an FMR/EPR frequency of MNPs in the bias field, and pickup coils or magnetometers measuring the magnetization induced in the MNPs by the bias fields and possibly the RF absorption. The computer derives MNP MPS/MSB spectra, magnetic particle images, or heats the MNPs using the EPR/FMR frequency field. A method of imaging or localizing the MNPs includes applying a magnetic field gradient; applying RF at an EPR/FMR frequency of the MNPs; sweeping magnetic bias field strength or RF frequency to sweep a resonance surface; applying RF at the EPR/FMR frequency, observing EPR/FMR resonances; rotating the magnetic bias field relative to the subject and resweeping the surface; and reconstructing a three-dimensional distribution of MNPs.

### CLAIM TO PRIORITY

The present application is a continuation in part of U.S. patent application Ser. No. 18/116,742 filed Mar. 2, 2023 which claims priority to U.S. Provisional patent application 63/315,626 filed 2 Mar. 2022. The entire contents of the aforementioned patent applications are incorporated herein by reference.

### BACKGROUND

Magnetic nanoparticles (MNPs) are nanoparticles of size 1-10³ nanometers diameter, and magnetic microbeads of size 10³-10⁶ nanometers diameter, which incorporate a magnetic core including at least one magnetic material. MNPs are of interest in biology and medicine because research has shown MNPs can be tagged or labeled with tissue-selective agents such as antibodies and other ligands. Concentrations of MNPs in tissue can be imaged or localized with several techniques thereby identifying tissues binding tissue-selective labeled MNPs. MNPs can be administered through catheters to specific tissue locations thereby forming MNP concentrations in those tissues. Further, MNP concentrations in tissue can be electromagnetically heated to destroy or damage tissue containing the MNPs. MNPs can also be tagged with antineoplastic agents and magnetically guided to specific tumor locations, and for other purposes. Localizing concentrations of MNPs in tissue is known as MNP imaging (MPI), MPI has been demonstrated with prototype machines.

Magnetic nanoparticles typically respond to applied magnetic fields by aligning with the magnetic field. When the magnetic field is removed, the magnetic nanoparticles relax to random orientation. The randomizing and aligning of the magnetic nanoparticles creates a signal that can be used to identify location and other characteristics of the nanoparticles.

When exposed to alternating magnetic fields, the alignment and randomization of magnetic nanoparticle orientation can cause heating of the nanoparticles and any tissue containing the magnetic nanoparticles.

Thermal therapies are common in clinical practice: 360,000 ablation procedures (RF, microwave, cryogenic and ultrasound) occur every year that cost $1.7B. Heating is also valuable as an adjunct to other therapies like radiation and chemotherapy (e.g., HIPEC). New thermal therapies are also actively being developed: perhaps the most promising applications are thermal drug release and immune stimulation.

Selectively releasing drugs from nanoparticle (NP)-liposome complexes that are antibody bound to cancer cells are possible. Very toxic drugs can be used because the local release of the drug limits systemic toxicity. For immune stimulation, heating MNPs bound to specific immune cells might provide better control of the immune response.

Magnetic MNP heating provides significant potential advantages over traditional ultrasound and RF methods, including being less invasive because vascular injection is possible instead of direct insertion of antennas or needles into a tumor as is necessary for RF or cryogenic ablation, and they are not limited by air and bone like ultrasound. MNPs can also be antibody targeted. However, MNP heating has two critical physical limitations: a) insufficient heat deposition at depth and b) inability to monitor temperature during treatment.

The limitations of heating through Brownian and Neel relaxation have been established and the promise of ferromagnetic resonance (FMR) heating has long been known and has been shown in Lee J-H, Kim Y, Kim S-K. Highly efficient heat-dissipation power driven by ferromagnetic resonance in MFe2O4 (FMR can increase heat deposition by at least two orders of magnitude over Neel relaxation or Brownian motion techniques. Clinical application of FMR has been difficult because For magnetic fields that are sufficient to align the spins, the resonant frequencies are very high, about 3 GHz, and have very high absorption in normal tissues.

Electron paramagnetic resonance (EPR), also known as electron spin resonance (ESR), involves interactions of electrons with an electromagnetic wave and occur at a frequency dependent on the magnetic field present at the resonating electron. EPR resonance is a phenomenon where unpaired electrons in materials resonate at specific frequencies (the Larmour frequency) in a static bias magnetic field. FMR is resonance of the magnetization of a magnetic material which can be thought of as EPR resonance of the free electrons forming the magnetization in the magnetic material; the largest difference between FMR and an EPR resonance is that the FMR resonance explicitly includes the impact of the collection of fields associated with the crystal structure of the magnetic material.

EPR resonance resembles, but is at very different frequencies from, nuclear magnetic resonance (NMR) of unpaired protons (typically hydrogen protons) commonly taken advantage of in NMR spectrographs used for chemical analysis and in magnetic resonance imaging (MRI) as used for medical imaging. EPR Larmor frequencies also increase with magnetic field strength.

In contrast to the FMR and ESR of free electrons in MNPs, a low frequency or static field produces a low frequency or static magnetization of the MNPs. Since signals generated by a low frequency magnetic field differ between bound and free MNPs, it is possible to distinguish concentrations of bound MNPs, such as MNPs bound in blood clots in vivo, or labeled MNPs bound to a biomarker or other ligand in vitro or in vivo, from unbound MNPs using a form of magnetic particle spectroscopy (MPS) termed magnetic spectroscopy of Brownian motion (MSB) because it characterizes Brownian rotation of the MNPs.

The signal detected in magnetic particle imaging (MPI) and magnetic particle spectroscopy (MPS) is generated by a change in direction of the magnetization of MNPs. The most robust way to increase the signal is increasing the size of the applied field which pushes the magnetization to change direction more swiftly increasing the signal. An alternative is to change the field more swiftly; i.e., increase the frequency of the applied field. Increasing the frequency works until the relaxation no longer allows the magnetization to change direction. Therefore, the signal in traditional MPI and MPS machines is fundamentally limited by the size and frequency of the magnetic fields used to manipulate the MNPs.

### SUMMARY

A nanoparticle heating system for cancer thermal therapies combines very low frequency alternating magnetic fields with pulsed RF fields. The pulsed RF is at the ferromagnetic resonant frequency of the nanoparticles to deposit energy into the nanoparticles very efficiently. The very low frequency alternating magnetic field algins the MNPs during the peak field and produce lower resonance frequencies during and around the null in the alternating field. The lower frequency resonance frequency mitigates heating of surrounding normal tissue allowing penetration through normal tissues.

Further, the MNP dynamics in an alternating magnetic field provides other mechanisms to reduce the resonance frequency. First, increasing the frequency of the alternating field increases the lag between the easy axis of the MNP and the applied field. As the lag increases, the vector sum of the applied field and the anisotropy field is reduced as they begin to cancel each other lowering the resonance frequency. In addition, manipulating the magnetization (orientation of the electron spins) and the easy axis separately introduces more possibilities to reduce the resonance frequency. Adjusting the anisotropy field and Neel relaxation time can manipulate the resonant frequency.

A magnetic nanoparticle (MNP) machine has magnets providing a bias field to a sample space; a pair of resonant drive coils bracketing the sample space; at least one pickup coil coupled to a lock-in amplifier, the lock-in amplifier coupled to provide signals to a computer; and a radio frequency (RF) stimulus coil driven at an electron paramagnetic resonance (EPR) frequency of MNPs in the bias field where the computer is configured to provide a MNP Brownian motion spectrum from the signals magnetic particle images from signals received from the lock-in amplifier.

### BRIEF DESCRIPTION OF THE FIGURES

FIG. 1 is a sequence diagram illustrating an effect of a pulse of radio frequency energy at an EPR or FMR resonant frequency on the magnetization of magnetic nanoparticles.

FIG. 2 illustrates the effect of the EPR resonant frequency pulse on magnetic nanoparticle ferromagnetic resonance Larmour resonant frequency.

FIG. 3 is a histogram of ferromagnetic resonance frequencies at points P and V in FIG. 2

FIG. 4 illustrates a coil arrangement for adding an EPR resonant frequency pulse to an existing MPS/MSB spectrographic machine.

FIG. 5A an 5B illustrate a pulse train used to measure magnetic particle heating from RF stimulation of ferromagnetic resonance in the existing MPS/MSB spectrography machine modified with the coils of FIG. 4.

FIG. 6 illustrates results of heating nanoparticles and monitoring temperature with MSB showing temperature rise is linear with applied RF power.

FIG. 7 illustrates a calibration curve of MSB second and fourth harmonics versus temperature that can be used to monitor temperature in magnetic nanoparticles driven by FMR heating.

FIG. 8 illustrates sense signals resulting when a magnetic field is swept through magnetic nanoparticle resonance with an applied electromagnetic field.

FIG. 9 is a schematic diagram of an MNP spectroscopy machine adapted to apply a radio frequency electromagnetic field to stimulate EPR resonance in nanoparticles.

FIG. 10 is a timing diagram for embodiments where both the EPR resonant drive and the FMR resonant drive is pulsed.

FIG. 11 is a schematic diagram of a MNP imaging (MPI) machine adapted to use a radio frequency electromagnetic field to stimulate EPR resonance in nanoparticles.

FIG. 12 is a schematic diagram of an alternative MNP imaging machine.

FIG. 13A is a simulation graph of nanoparticle magnetization of MNPs in an MPI machine where MNPs are located near a peak magnetic field of a magnetic field gradient, and FIG. 13B is a simulation graph of detectable signals produced by the MNPs of FIG. 13A.

FIG. 13C is a simulation graph of nanoparticle magnetization of MNPs in an MPI machine where MNPs are located centrally in a magnetic field of a magnetic field gradient, and FIG. 13D is a simulation graph of detectable signals produced by the MNPs of FIG. 13C.

FIG. 14 illustrates a machine for heat-treating abnormal tissue in a subject where heating of the subject is localized to a surface within the subject where MNPs in an applied magnetic field have FMR resonance with an applied RF field upon an EPR pulse with mechanical rotation.

FIG. 15 illustrates a machine for heat-treating abnormal tissue in a subject where heating of the subject is localized to a surface within the subject where MNPs in an applied magnetic field with electronic rotation have FMR resonance upon an EPR pulse with an applied RF field. The EPR coils are not shown for simplicity.

FIG. 16 is a flowchart of a method of generating an enhanced image of targeted magnetic nanoparticles in a subject by imaging targeted magnetic nanoparticles, imaging nontargeted nanoparticles, and subtracting the nontargeted image from the targeted image.

FIG. 17 is a flowchart of a method of using two types of MNPs, one targeted to a tumor, and a second nontargeted, to enhance imaging of the tumor.

FIG. 18 illustrates resonance at an LF-AMF frequencies when pulses at EPR resonance frequencies are applied or are not applied to samples either containing water or MNPs.

FIG. 19 illustrates signal at 2^{nd} and 4^{th} harmonics of LF-AMF resonance frequencies versus applied RF power at the EPR resonance frequency of a sample of MNPs.

### DETAILED DESCRIPTION OF THE EMBODIMENTS

When unpaired electrons of a magnetic nanoparticle (MNP) are subjected to an applied magnetic field, they tend to be aligned by that magnetic field as time progresses. The signal detected in magnetic particle imaging (MPI) and magnetic particle spectroscopy (MPS) is generated by a change in direction of the magnetization of MNPs.

The most robust way to increase the signal in MPI and MPS is increasing the size of the applied field which pushes the magnetization to change direction more swiftly increasing the signal. The alternative is to change the field more swiftly; i.e., increase the frequency of the applied field. Increasing the frequency works until the relaxation no longer allows the magnetization to change direction sufficiently swiftly. Therefore, the signal is fundamentally limited by the size and frequency of the magnetic fields used to manipulate the MNPs.

A new method of manipulating the MNP magnetization uses an additional electromagnetic (EM) field, generally in the radio-frequency (RF) range, at the resonant frequency of the free electrons some of which produce the magnetization. We term that RF as the “EPR field”, “ESR field,” or similar. The EPR field can help speed up the alignment of the MNP magnetization to an applied field or it can eliminate the MNP magnetization either temporarily or permanently.

When unpaired electrons of a MNP having magnetic domains aligned with or against a magnetic field interact through EPR resonances with an applied electromagnetic field, the up and down spin states of those electrons are flipped impacting the MNP magnetization by
1) tipping or even reversing the MNP magnetization or,
2) for higher power, equalizing the up and down spin state, thereby eliminating the magnetization of the MNPs.

When the magnetization of the MNPs is eliminated by the EPR resonance, upon termination of the EPR field the electrons forming the MNPs magnetization relax returning the MNP magnetization to its original size and, over time, the magnetization once again becomes aligned by the low frequency or static magnetic field.

The realignment of MNPs with the magnetic field gives a detectable signal that varies in magnitude with quantity of the electrons realigning with the magnetic field and a time derivative of the changing magnetization.

We use these methods to improve MPI, MPS, MSB and MNP hyperthermia treatment. The slower magnetic fields used in MPS and MPI, the LF-AMF, are used in combination with the EPS RF field to improve MPS and MPI. We can also reduce the amplitude of the applied fields from traditional MPI by using the EPR/FMR resonance. There are also possibilities to design better MNPs, separate the signal from two or more different MNPs recorded simultaneously, or produce other mechanisms to localize the MNPs.

This method operates using the principle that when Neel and Brownian relaxation are too slow to allow the magnetization to change, a low frequency alternating magnetic field (LF-AMF) alone produces no signal. However, if an EPR resonant RF field is added when the applied LF-AMF is opposed to the magnetization, the magnetization will flip producing a signal that is uniquely produced by the combination of the LF-AMF and the EPR field. By varying the amount and timing of each, the signal will change allowing localization as well as relaxation estimation.

The LF-AMF accomplishes two functions: a) it aligns the MNP magnetizations and in doing so it aligns the free electron spins and b) creates the Zeeman splitting that enables the resonant absorption. As the LF-AMF drops to zero, relaxation keeps the MNP magnetizations aligned but the Zeeman splitting is reduced, reducing the resonant frequency.

The magnetic field the unpaired electrons feel is the combination of the LF-AMF and the internal fields of the crystal much like the susceptibility in MRI. We use the term “anisotropy field” to denote the combination of the intrinsic fields in the crystal. The resonance frequency is smallest when the LF-AMF is in the opposite direction as the anisotropy field so the Zeeman splitting is the difference between the two.

If the relaxation (the combination of the Brownian and Neel) is known, the time when the LF-AMF and the anisotropy field are opposed can be calculated. The resonant frequency can also be calculated.

Further, the anisotropy and relaxation can be designed to vary with different MNP types enabling a different signal to be produced by each MNP type because the resonant frequency and timing is different.

In addition, the signal generated by a relatively small applied LF-AMF can be enhanced to produce a large change in magnetization using an EPR field. If a large applied magnetic field (either alternating or DC) aligns the easy axes of the MNPs and is then replaced by a small LF-AMF, no signal is generated till an EPR field allows the small applied field to flip the magnetization.

Further, the EPR signal from the electrons in the MNPs can be recorded and combined with the lower frequency signal from the changing MNP magnetization to gain signal and allow combinations of imaging and sensing techniques.

The rate at which the MNPs easy axis alignment is reduced can be measured by waiting to sample the signal with the combination of the LF-AMF and EPR field. For large MNPs, the rate is the Brownian relaxation which couples the MNP to the microenvironment and allows measurement of binding, viscosity, temperature, pH, matrix rigidity, cell uptake, inflammation, etc. The change in size and shape of the signal peaks can be analyzed to provide that information. For smaller MNPs the magnetization can be rotated separately from the easy axis which is governed by Neel relaxation.

### FMR Heating of Nanoparticles Using EPR Pulses

We can increase energy deposition into magnetic nanoparticles (MNPs) beyond that typically seen using EPR or ferromagnetic resonance (FMR) magnetic field pulses. FMR can increase heat deposition by at least two orders of magnitude over Neel relaxation or Brownian motion techniques. Clinical application of FMR has been difficult because:
1) the very high RF frequencies required, with typical magnetic fields about 3 GHz, have very high absorption in normal tissues and
2) the MNPs must be fixed in the correct orientation to the radio frequency (RF) fields.

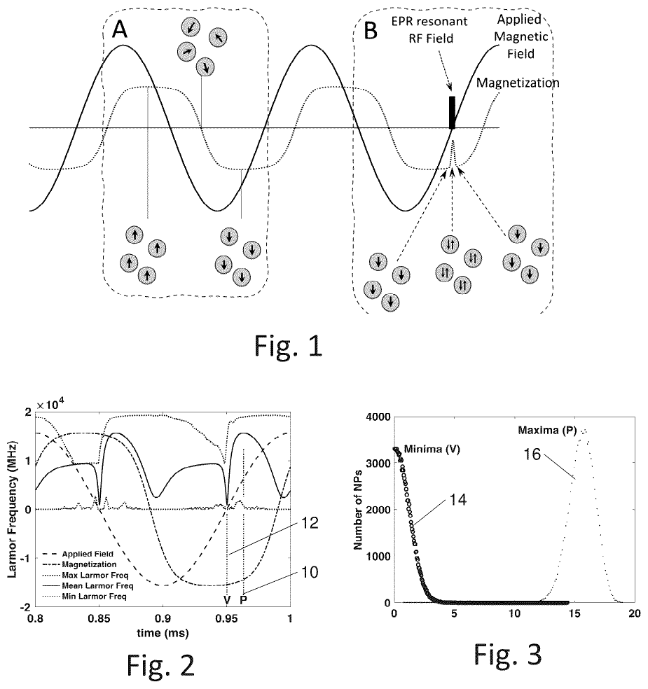

These limitations are illustrated in FIG. 1 where 3 GHz has been typically used. It is desirable to reduce the frequency used for FMR resonant heating to reduce heating of adjacent tissues.

We use a mechanism for overcoming both of these limitations that can also allow the heat deposition to be macroscopically localized using magnetic field gradients.

To improve power dissipation in the MNPs, and to assist in locating the MNPs, we use a low frequency (kHz) alternating magnetic field (LF-AMF) to i) align the MNPs during the peaks and ii) reduce the FMR resonance frequency during the zero-crossings. FIG. 1 illustrates the concept by portraying a pulse sequence where an applied burst of RF radiation at an EPR resonant frequency reduces magnetization of the nanoparticles, which then allows a low frequency FMR resonance to occur.

TABLE 1
The NP energy absorbed and tissue eddy current energy absorbed as
a function of changes in FMR resonant frequency and RF power.

| | | | | Tissue |
|---|---|---|---|---|
| FMR Resonant | Change in | Change in | NP Absorbed | Absorbed |
| Frequency | Frequency | RF Power | Energy | Energy |
| 3 GHz | 1 | 1 | High | Very High |
| 30 MHz low power | [symbol] 10⁻² | 1 | 10⁻² | ×10⁻⁴ |
| 30 MHz high power | 10⁻² | [symbol] 10² | High | ×10⁻² |

In FIG. 1, The A box (left), shows how the magnetization (dashed curve) reflects the alignment of the MNP easy axes. The LF-AMF (kHz) (solid curve) pulls the magnetization which in turn pulls the MNPs' easy axes into alignment with the LF-AMF. There is a lag in the ability of the MNPs to follow the LF-AMF. The lag is characterized by the relaxation time. Relaxation keeps the MNPs aligned after the LF-AMF changes sign so when the applied field is zero, the MNPs remain aligned. The LF-AMF can be changed to adjust the lag for a given relaxation.

In FIG. 1, the B box (right), shows the results of an RF pulse on the magnetization when the applied field is close to zero. The net field is small, so the resulting Larmor frequency is low. The RF absorption is very high at resonance and the resonance frequency is very low because the misalignment of the magnetization, the anisotropy field and applied field makes the effective field small.

The frequency of the LF-AMF is selected so the relaxation induced phase lag leaves the MNPs aligned during the zero-crossing. The MNPs are heated by a pulsed RF field near the LF-AMF zero-crossing when the FMR resonance frequency is low, so MNP absorption of the RF is high, and tissue absorption is low. The temperature of the MNPs, and hence temperature of associated tissue, can be monitored using the LF-AMF induced MNP magnetization.

FMR produces at least two orders of magnitude larger absorption than the Brownian/Neel mechanisms. However, FMR has only been observed at high frequencies where tissue heating is prohibitive; the work of Lee was done at 3 GHz where FMR research is generally performed, and which is a frequency near those used for microwave ovens. Our long term objective is to heat MNPs selectively through FMR with low tissue heating. Lower FMR resonant frequency reduces tissue heating dramatically; the energy per FMR electron transition is also reduced but the number of transitions can be increased to compensate for the energy loss effect (see Table 1).

Tissue heating is dominated by eddy current heating, is characterized by SAR and is proportional to ω2·PRF where ω is the frequency and PRF is the RF power. The energy absorbed with MNP FMR is the photon energy, ℏω, times the number of photons absorbed (i.e., the RF power, PRF). Therefore, reducing the FMR resonance frequency would drop tissue heating much more than it would drop MNP heating. For example: the very high MNP heating Lee et. al. achieved at 3 GHz can be attained with much lower tissue eddy current heating if the FMR resonant frequency can be reduced. If the FMR resonance frequency is dropped by a factor of 102 (down to 30 MHz) with the same RF power, the tissue heating drops by a factor of 104 and the energy absorbed by the MNPs drops by 102. If the RF power is then increased by 102, the energy absorbed by the MNPs would return to the level achieved at 3 GHz, but the tissue heating would be a factor of 102 lower.

### NP FMR at Low RF Frequencies

Improving MNP heating has a host of applications starting with more localized ablation methods and includes improved thermal drug release. Thermal drug release that is localized to a cancer allows drugs or drug dosages that are too toxic for systemic treatment to be used because the local release limits systemic toxicity.

Another goal is only heating bound MNPs enabling selective drug release from liposomes that are bound to cancer cells. FMR has its roots in the physics literature beginning in the 50's and extending to the present. The most FMR heating appeared in a recent report of extremely high energy deposition achieved by aligning the easy axes of the MNPs and rotating the magnetization at the Larmor frequency, but the FMR Larmor frequency was too high for in vivo use.

### MNPs are Heated Using Ferromagnetic Resonance at Low Enough Resonant Frequencies to Mitigate Heating of Surrounding Normal Tissue

NP dynamics and FMR are complicated. We developed codes to describe resonance during MNP motion in a time varying magnetic field. FMR requires the net magnetic field that the unpaired electrons in the MNPs feel because the net magnetic field at each nanoparticle determines the Larmor resonance frequency associated with that MNP. The net field is the vector sum of the applied field and the local fields produced by the crystal and its surroundings that are generally lumped together and termed the anisotropy field. FMR simulations describe the electron dynamics but generally assume a stationary crystal in a static magnetic field38; they do not attempt to describe the resonance conditions during MNP movement.

Our simulations use the Langevin equation (LE) for the magnetization. The LGL equations describe MNP motion in a LF-AMF.

$$\frac{d \overline{M}}{d t} = \frac{( \overline{M} \times \overline{ξ} ) \times \overline{M}}{2 {τ}_{B}} + \frac{{\overline{λ}}_{t} \times \overline{M}}{\sqrt{{τ}_{B}}}$$

Where the magnetization is determined completely by two unitless numbers: the unitless frequency, Ω=fτ_{B}, and the unitless field:

$$ξ = \frac{μ H}{{k}_{B} T} .$$

The variables involved are: f is the frequency of the LF-AMF, τ_{B} is the Brownian relaxation time, μ is the MNP magnetic moment, H is the amplitude of the LF-AMF, k_{B} is the Boltzmann constant, and T is the temperature.

### Anisotropy

The anisotropy field makes FMR different from electron paramagnetic resonance (EPR) or nuclear magnetic resonance (NMR).

FIGS. 2 and 3 show one way the FMR Larmor frequency is lowered by a LF-AMF in simulations that are limited to Brownian relaxation where the magnetization is locked to the easy axis of the crystal. In FIG. 2, a simulation, made using just Brownian relaxation, illustrates the ability of LF-AMF to reduce the FMR Larmor frequency, ω_{L}=γB_{eff}. A LF-AMF rotates the MNPs and the associated magnetization. If the LF-AMF (in the figure) changes too fast for the magnetization (in the figure) and the easy axis to keep up, a lag occurs where the magnetization lags the LF-AMF. The lag between the easy axis and the LF-AMF reduces the FMR resonance frequency. We simulated 10⁵ MNPs in a 5 kHz, 10 mT LF-AMF and calculated the FMR resonant frequency at each timestep. The mean Larmor frequency is plotted with a solid line and the maximum and minimum in the distribution are plotted with dotted lines. Minima occur at the zeros of LF-AMF and magnetization. The overall minima is when the LF-AMF passes through zero. Maxima and minima in the mean Larmor frequency are marked with “P” and “V”.

The distributions of the frequencies are represented by the histograms of the Larmor frequencies for times marked V 12 and P 10 in FIG. 2 are displayed in FIG. 3 with time V 14 and time P 16. The RF power absorbed is estimated from these. For P, it is centered at 15.9 GHz. For V, most of the MNPs have FMR resonant frequencies of essentially zero. By manipulating the dynamics of the MNP population with a LF-AMF we can move the FMR resonance frequency to very low values.

Higher LF-AMF frequencies produce more lag between the LF-AMF and the easy axes reducing the average Larmor frequency.

Including manipulation of the magnetization relative to the easy axis of the MNP characterized by Neel relaxation enables more possible mechanisms to reduce the Larmor frequency to be used.

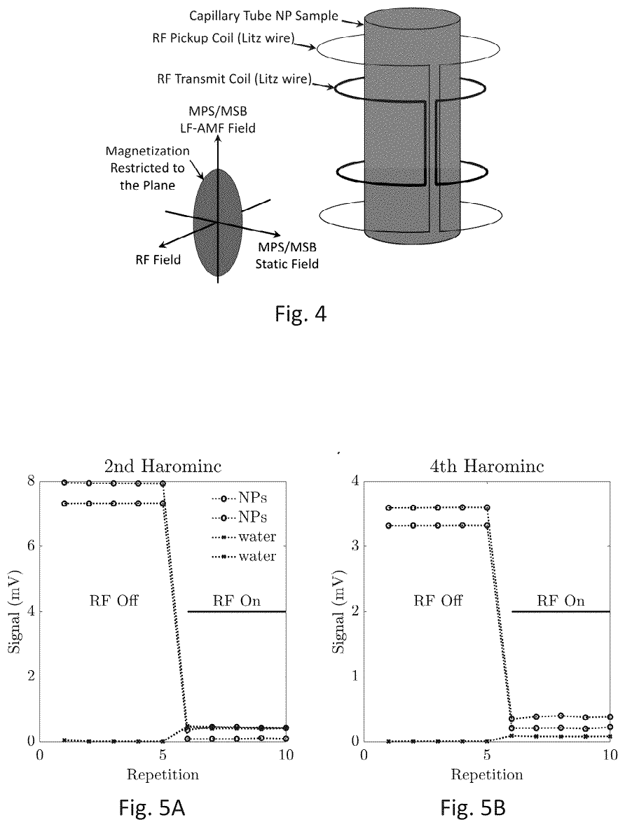

An EPR add-on coil for an existing magnetic particle spectroscopy MPS/MSB magnetic particle spectroscopy of Brownian motion machine is illustrated in FIG. 4. The RF pickup coil measures the field produced by the transmit coil that is not absorbed by the MNP sample, providing an EPR-like measure of RF absorption which shows FMR absorption. The cylindrical RF coil system is inserted into the MPS/MSB spectrometer, as illustrated in FIG. 9, with its EPR RF coils oriented perpendicular to both the direction of the MPS/MSB static field and the MPS/MSB LF-AMF fields.

An MPS/MSB spectrometer produces the LF-AMF field and measures the magnetization used to estimate temperature during heating. The capillary tube sample is placed in the center of the sample space. The saddle shaped RF transmit coil surrounds the sample. The RF pickup coil is also a saddle shaped coil located outside the transmit coil as shown. The sample is thermally isolated by an air gap between it and the RF coils. The RF coils are cooled via the MPS/MSB coil cooling system. The capillary tube sample is suspended in the center of the RF coils and held in place with clamps on the ends of the tube.

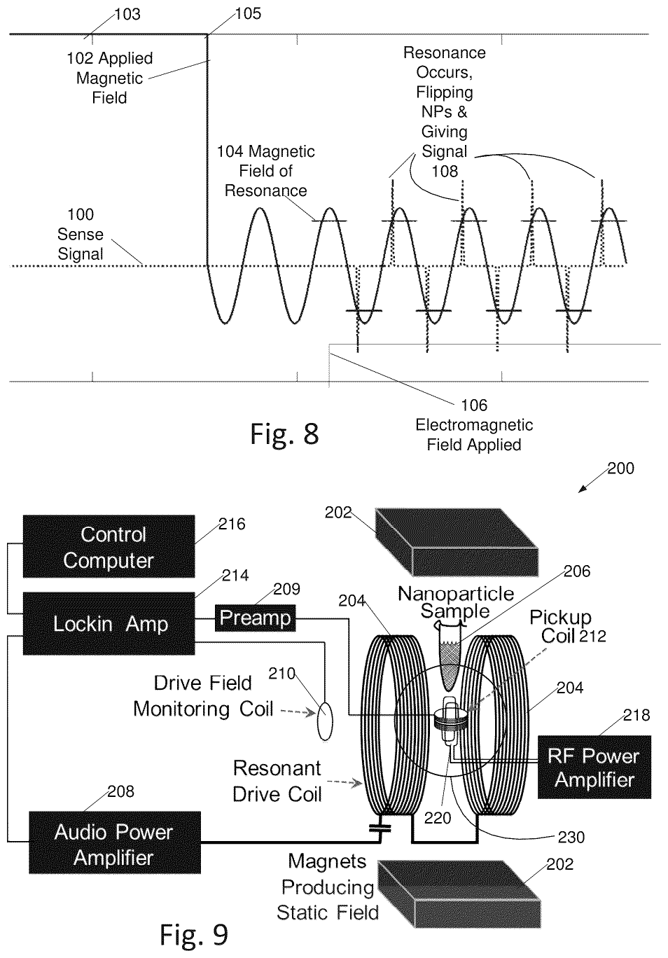

A 50 MHz (Zurich HF2LI) lockin amplifier generates the RF. The signal is amplified using an LZY-22+30 W RF amplifier (0.1-200 MHz) from Mini-Circuits. The signal from the RF pickup coil is measured with the LZY-22+ lockin. The magnitude of the first harmonic reflects the amount of energy absorbed by the sample; it is essentially a low frequency EPR system. In embodiments, a Zurich MFLI 500 kHz lockin amplifier is used for the MPS/MSB LF-AMF. The MFLI lockin has a digital recording option so we can record the raw waveform as well as the harmonics to estimate both the RF absorption as in FIG. 5 and the temperature of the MNP sample as in FIG. 8. The LF-AMF and RF frequencies and timing are set to maximize the energy deposition 30 MHz to 40 MHz FMR resonant frequencies.

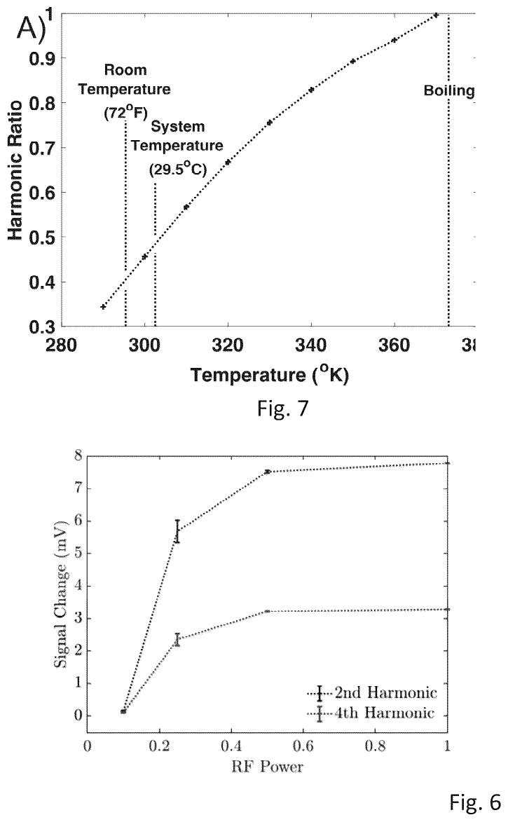

FIGS. 5A, 5B, and 6 demonstrate FMR absorption in a MNP sample using LF-AMF and <low frequency RF. Two MNP samples and two water samples are shown with RF off for 5 repetitions followed by RF on for 5 repetitions. The MPS/MSB spectrometer used a 2241 Hz LF-AMF, 10 mT alternating magnetic field with a 1 mT static perpendicular field. The RF was generated with a prototype coil at 31 MHz at 0.5 W. The FMR absorption caused the large 7.6 mV 2nd harmonic signal (shown in FIG. 5A) to drop to background (0.3 mV) when the RF was turned on. Similarly, the 3.5 mV 4th harmonic (shown in FIG. 5B) also dropped to background when the RF was turned on. A 1 kHz LF-AMF produced no change in measured harmonics indicating no resonance was observed for low frequencies where the magnetization did not lag the applied field sufficiently.

FIG. 6 specifically illustrates change in MPS/MSB measurements monotonically by RF power showing that the MPS/MSB signal change is monotonic with RF absorption and thus with heating. MNP samples were measured with and without RF. The decrease in signal is plotted vs RF power. The RF frequency was 31 MHz. Each value is the mean of the differences between five measurements without and with RF. No signal change was observed for higher frequencies (up to 400 MHz).

To add perspective, the data shown in FIG. 5A and 5B, were generated using 0.5 W and a typical MRI uses a 35 kW RF amplifier. MRI systems generally only hit the 0.4 W/kg SAR limit for MRIs on 3T scanners which function at 128 MHz. At 32 MHz, 16 times the power could be used before the MRI SAR limit would be reached. Even accounting for differences in mass, the RF power used for the data shown in FIGS. 5A-5B would not reach the MRI SAR limits for eddy current heating in normal tissue. Further, limiting a therapy procedure to the MRI SAR limits is not required. Driving RF at the lowered FMR frequency can thus heat MNPs without heating normal tissues in human bodies at low RF frequencies.

### Measuring Temperature Increases Through MSB

We use a system derived from magnetic nanoparticle spectrography of Brownian motion to estimate the temperature during heating of tissue using a calibration curve. To obtain a raw calibration curve, an MNP sample is placed in a water bath at a set temperature till it reaches equilibrium. The MNP sample is then placed in the spectrometer for MPS/MSB measurement. The sample had an air gap around it so it maintained its temperature long enough for MPS/MSB measurements. A baseline signal was measured before heating, and we used changes from that baseline to estimate changes in temperature via the calibration curve as shown in FIG. 7. The measurements in water should be very accurate. For in vivo use where the viscosity is not known, the calibration curve includes an estimate of the viscosity as well. We also evaluate errors caused by ignoring the effects of baseline viscosity variation which are relatively small for the smaller temperature changes that occur in applications like drug release and hyperthermia. The effects of MPS/MSB are larger for ablation where temperature changes are larger. Fortunately, ablation requires less precision in temperature. Simulations are used to estimate these potential errors and adjust the calibration curves accordingly. In an embodiment, the computer is configured to use the magnetic spectra of Brownian motion to map temperature in 3 dimensions throughout tissue.

In another embodiment bound MNPs are selectively heated (without significant heating of unbound MNPs). Find the relaxation of the nanoparticle aggregates to identify the correct timing for the LF-AMF to produce an EPR/FMR resonance. Several binding variations are possible: antibody binding to targeted molecules or to specific cells. We would use an LF-AMF (MPS/MSB) frequency and RF frequency tuned to produce a resonance for bound MNPs.

In another embodiment the LF-AMF-induced reduction of the EPR or FMR resonance frequency is taken advantage of during RF-heating of MNPs located within a tumor or other abnormal tissue that is to be heat-treated. High temperatures applied to tumor tissue can destroy that tumor tissue, and that while heating tumor tissue it is desirable to minimize heat applied to nearby normal tissue. If the RF energy applied at the EPR or FMR resonance frequency is intense enough, the temperature of tissue around the MNPs increases allowing either hyperthermia or ablation of the tissue.

The location that is heated can be controlled by localizing the RF energy using location specific antennas or coils. The location can also be controlled by using static or dynamic magnetic field gradients to produce different resonance frequencies at different locations. The resonant frequency can be selected to deposit energy at desired locations along the gradient. An alternative is to use LF-AMF fields that are different magnitudes at different locations.

Magnetic fields can be generated that vary with location producing resonance along selected surfaces in the patient and at no other locations. By pulsing the EPR or FMR frequency radiation to match the resonance along selected surfaces in the patient, or by pulsing the RF at the EPR or FMR resonance to produce resonance along selected surfaces in the patient as determined by the applied magnetic fields. The increased absorption of RF energy at FMR resonance allows the RF field to be low enough energy to penetrate other tissue of the subject without significant absorption. Mapping temperature through the tissue as described in the previous paragraph is done to monitor temperatures applied to both the abnormal or tumor tissue and adjacent tissues.

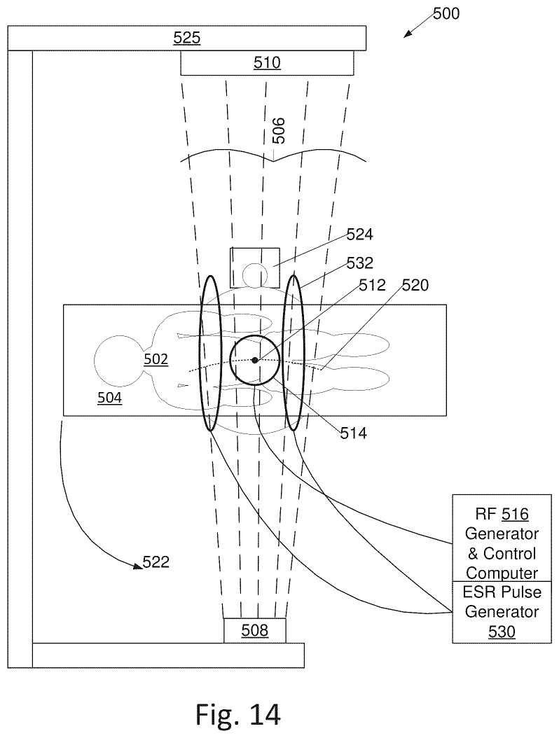

In a machine 500 (FIG. 14) for heat-treating abnormal tissue in a subject 502 or other biological material, the subject or biological material is placed on a rotatable table 504 in a treatment space having a magnetic field 506 with an intensity gradient, the field being produced by magnets 508, 510 that may be coupled by a magnetic core 525. The rotatable table 504 is rotatable about an axis 512, and an RF heating coil 514 is positioned over the abnormal tissue (not shown for simplicity) in subject 502. When RF power is provided by a pulsed RF source 530 to EPR coils 432 at the EPR/FMR frequency, nanoparticle magnetization is disrupted. If RF power is then provided by a RF generator and control computer 516 to RF heating coil 514, EPR/FMR resonance occurs in MNPs within subject 502 or biological material along a volume 520 determined by the magnetic field strengths and the magnetic field gradient; in a particular embodiment the magnetic field is configured so surface 520 intersects the axis 512, and the subject 502 is positioned so the abnormal tissue is also located at the axis 512. While the subject 502 to the RF field produced by RF heating coil 514, the subject may be rotated 522 about axis 512 by a rotator motor 524 to reduce heating concentrations in normal tissue while continually heating abnormal tissue positioned at axis 512. In embodiments, the volume to be heat treated about the axis 512 may be increased by modulating magnetic field strength.

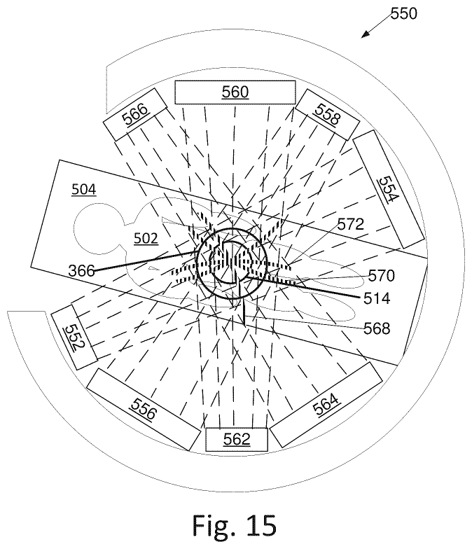

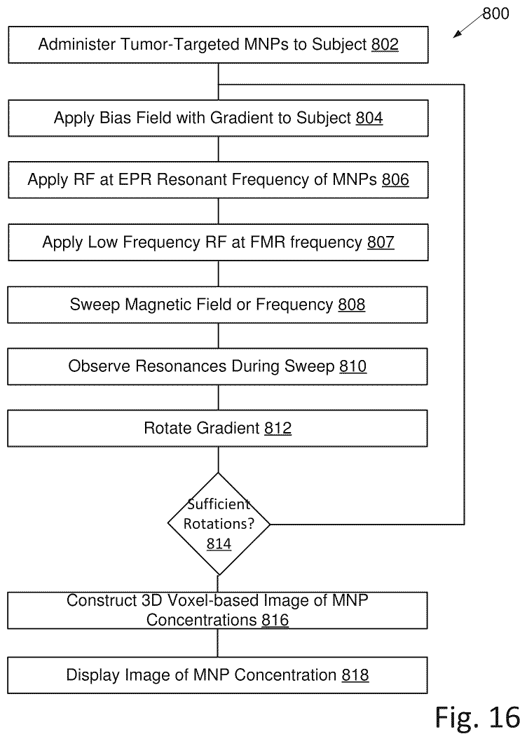

In an alternative embodiment 550, FIG. 15, instead of mechanically rotating the subject 502 relative to the magnetic field, the magnetic field is effectively rotated relative to the subject while RF heating coil 514 is energized by an RF generator and control computer (not shown in FIG. 5B for simplicity). This can be done by providing multiple sets of magnets, such as magnet pair 552, 554, magnet pair 556, 558, magnet pair 560, 562, and magnet pair 564, 566 and operating the magnet pairs sequentially. Similarly, multiple pairs of EPR resonance coils (not shown for simplicity) are provided, with appropriate drive electronics. For example, magnet pair 552, 554 may be operated to cause heating along surface 568, then magnet pair 552, 554 is turned off and magnet pair 556, 558 is operated to cause heating along surface 570, then magnet pair 556, 558 is turned off and magnet pair 560, 562 is operated to cause resonance and heating along surface 572, and so on; after all magnet pairs are operated the cycle repeats. By operating magnet pairs simultaneously even more angles of magnetic field gradient can be obtained. Thermal inertia of tissue allows cumulative heating of abnormal tissue at the intersection of surfaces 570, 572, 568 to effective hyperthermia treatment levels, while tissue elsewhere remains at lower temperatures even in a single resonance surface or volume 572.

In a particular embodiment, the MNPs are complexed with antibodies to a specific abnormal tissue type, such as a tumor, such that the MNPs concentrate in tissue of that specific abnormal tissue type. In this embodiment, the RF energy preferentially heats tissue the specific abnormal tissue type within which the MNPs concentrate.

The increased MNP relaxation resulting from the MNP binding the target cells or structures can be used to isolate their signal from that of the free MNPs and heat them preferentially.

### Applications to Magnetic Nanoparticle Imaging

In an MNP imaging system, a magnetic field 102 (FIG. 8) is applied to materials, which may include biological materials or a human, which contain MNPs. Initially, at time 103, this magnetic field is a high static bias field to which spins of the MNPs align. At time 105, this magnetic field is reduced in intensity and begins alternating, as the magnetic field alternates it passes through a magnetic field intensity 104 at which resonance occurs with a particular radio frequency (RF) of electromagnetic radiation that is applied at time 106. When that particular frequency of electromagnetic radiation is applied and the magnetic field strength passes through the magnetic field intensity 104 at which resonance occurs, resonance causes the aligned spins to flip or randomize as resonance ceases these spins realign and give a detectable signal 108 in a pickup coil. Without resonance, sense signals 100 are flat. Where a biasing magnetic field gradient exists, the resonance occurs only when total magnetic field at the MNPs is such that the electron spins of the MNPs resonate at the particular applied frequency; by determining when this resonance occurs, we can determine a surface or volume where in the magnetic field gradient the MNPs must be located.

In principle, either the magnetic field intensity or the frequency of the applied

electromagnetic field may be swept through resonance to localize MNP concentrations in the biasing magnetic field gradient. With each direction of magnetic field gradient, as field strength or frequency is swept resonance of MNPs occurs along a surface in a three-dimensional imaging space; by changing direction of the magnetic gradient we change an angle of a surface of resonance and further localize the MNP concentrations as being at intersections of surfaces of resonance; by using a sequence of three or more noncoplanar magnetic field gradient directions with swept magnetic fields or frequencies we can localize and quantify MNP concentrations to points in a three-dimensional imaging space.

The MNPs can be imaged by measuring the RF absorption as the magnetic field gradient is moved across the subject or object to be imaged. The absorption decreases more for larger numbers of MNPs at the location or locations that the RF frequency matches the EPR/FMR frequency. By rotating and translating the gradient field, the MNP number at each location can be found using a Radon transform or modified Radon transform.

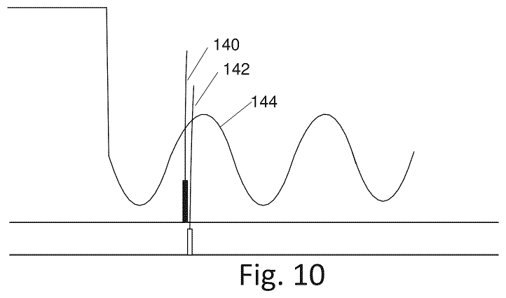

In an embodiment taking advantage of the lower FMR/EPR frequencies achieved when RF pulses at resonant frequencies are applied, a pulse of EPR resonant frequency radiation is applied 140 (FIG. 10) through EPR coils 230 (FIG. 9) as the applied magnetic field 144 gradient passes through a particular EPR resonance frequency at a particular location within tissue, the pulse 140 of radio frequency (RF) energy at the EPR resonance frequency 140 reduces nanoparticle magnetization at that particular location within the tissue briefly. A pulse 142 of an FMR resonant frequency RF energy determined by prior measurement or by the simulations previously described is applied either concurrently with or immediately after the pulse 140 of EPR RF and a signal is detected by a pickup coil. indicating resonance at the FMR frequency. Location is determined from intersecting surfaces where resonance at FMR frequency is detected as the magnetic field gradient is rotated relative to the nanoparticles in the subject.

In an alternative embodiment, a machine as described in the above paragraph is used to EPR/FMR resonant frequency radiation is first used to image MNP concentrations in the subject, confirming frequency and timing of the EPR/FMR resonance, then pulses 148 of high power EPR/FMR resonance frequency RF are applied at times relative to the magnetic field when EPR/FMR resonance was detected during imaging. The high power EPR/FMR resonance frequency RF is pulsed to reduce heating of normal tissues.

In a MNP spectroscopy machine (MPS/MSB) 200 (FIG. 9) adapted to apply an RF electromagnetic field to MNPs in a biasing magnetic field, magnets 202 provide a bias field to a sample 206; magnets 202 are magnetically coupled together by an iron core (not shown). A pair of resonant drive coils 204 bracket sample 206, the LF-AMF resonant drive coils 204 are driven by an audio amplifier 208. The drive magnetic field provided by the resonant drive coils 204 is sensed by a drive monitoring coil 210 and at least one, and preferably paired, pickup coils 212 oriented at right angles to the resonant drive coils 204 provides responses of MNPs in sample 206 through a preamplifier to a lock-in amplifier 214.

In an alternative embodiment, bias field magnets 202 are omitted with LF-AMF resonance drive coils 204 carrying sufficient current, which may be a DC current superimposed on low frequency AC currents, to provide enough total magnetic field that an EPR resonance can occur with applied RF fields in coil 220.

In some embodiments the pickup coils 212, which form part of an LF-AMF detection module, include a balancing coil configured to null signals produced in the LF-AMF detection module pickup coils in absence of MNPs in the sample space and thus provide better resolution of resonances in the MNPs.

Signals from lock-in amplifier 214 are digitized and provided to a computer 216 configured to provide a MNP Brownian motion spectrum from the signals. Computer 216 also controls operation of the system, including RF sources, audio sources, magnets, and resonant drive coils 204. A radio frequency signal is provided by an RF amplifier 218 at a resonant frequency. In this embodiment, an LF-AMF generated by the resonant drive coils 204 and the RF signal is applied through RF stimulus coil 220 by RF amplifier 218.

To take advantage of the EPR/FMR resonance frequency reduction upon application of an LF-AMF pulse herein described, an additional EPR/FMR coil 230 such as is described with reference to FIG. 4 is added to the MPS/MSB machine 200 and driven with appropriate electronics (not shown). When driven with a pulse sequence as described with reference to FIG. 10, this machine observes the reduced-frequency EPR/FMR resonance associated with the nanoparticles.

Static magnetic fields as described herein are produced by magnets that may be permanent magnets, electromagnet coils, or combinations of permanent magnets and electromagnet coils. Where magnetic fields are disclosed as having gradients, they are also produced by magnets that may be permanent magnets, electromagnet coils, or combinations of permanent magnets and electromagnet coils. Where magnetic fields are disclosed as being swept in intensity these fields may be produced by either electromagnet coils or by a combination of permanent magnets and electromagnet coils.

When resonance occurs, MNP magnetization alignment is disturbed in sample 206 and a signal is picked up by pickup coils 212. Timing of the signal relative to the audio signal waveform and pulses applied with the EPR coil, as analyzed by computer 216, provides a MNP spectrum by identifying the time lag before the MNPs reach resonance.

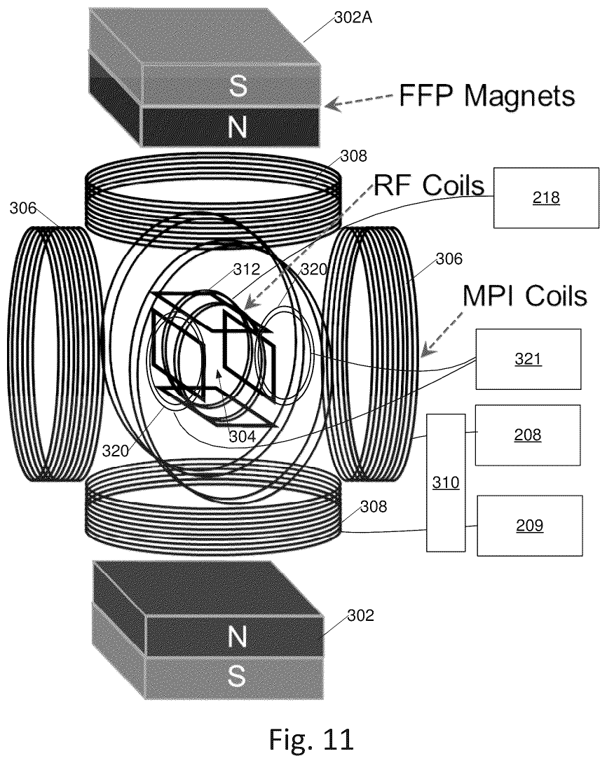

In a MNP imaging (MPI) embodiment 300 (FIG. 11), permanent magnets 302 (or equivalent coils) provide a magnetic field with a set of magnetic field gradients that can be moved using the “MPI coils” 306, 308, 309 across a space 304 within which a subject or biological sample (not shown in FIG. 11) may be positioned. The current in the MPI coils (306, 308, 309) is driven by an audio amplifier 208. As the gradients pass over MNPs, the MNPs flip orientation producing a signal in pickup coils 312 (only one of the three pairs of coils are shown in FIG. 11) amplified by a preamp and sampled by an analog to digital converter 209. Alternatively, the “MPI coils” 306, 308, 309 can be used to both drive the gradients and detect the signal using a switch 310 to direct the signal to the preamp and converter 209. Space 304 is also surrounded by RF coils 312 coupled to be driven by RF amplifier 218. Again, to keep the figure readable, only one of three pairs of RF coils are shown. The combination of RF coils excited determine a direction of the RF field produced. The orientation of the coils producing the magnetic fields (302 and 308, 309, 309) determine the direction of the applied field. A set of EPR-pulse coils 320, not found in traditional MPI machines, are driven by a EPR pulse driver 321. The MNPs located where the magnetic fields and the RF fields produce an EPR resonance absorbs the EPR pulse and has their magnetization reduced, these magnetic nanoparticles then absorb RF at the FMR resonant frequency and flip orientation. Additional electronics similar to that of FIG. 8 is provided but not shown in FIG. 11 for simplicity, including lock-in amplifier 214 or other amplifier/analog to digital conversion unit, is coupled to computer 216. The computer 216 is configured to perform magnetic particle imaging based on the signals it receives from the lock-in amplifier. Operation is similar to the embodiment of FIG. 8 in that timing of the signal relative to the audio signal waveform, as analyzed by computer 216, for both the EPR pulse and the FMR sensing, is determined, this timing however provides not a spectrum but a surface on which the MNPs are located within the subject or biological sample in space 304. Additional imaging may be obtained by switching operation of the drive and sensing coils by switching switch 310.

Imaging in the embodiment of FIG. 11 is performed by using the MPI coils 306, 308, 309 as electromagnets on the main magnets 302 to scan the magnetic field gradients through space 304 and across any subject bearing MNPs while providing an audio-frequency AC field in the drive coils on top of the main field and recording and analyzing signals received by the sense coils, gradients are scanned to identify locations of the MNPs. In alternative embodiments, the sense coils form part of an LF-AMF detection module and may be supplemented or replaced in such an LF-AMF detection module by other forms of magnetic field sensing devices or magnetometers, such as but not limited to Hall-effect sensors, superconducting quantum interference devices (SQUIDs), and magneto-optic Kerr-effect magnetometers.

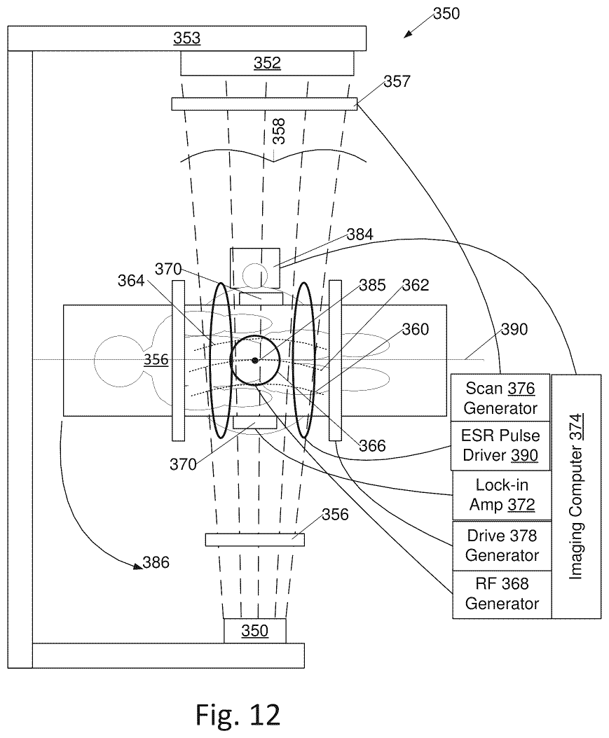

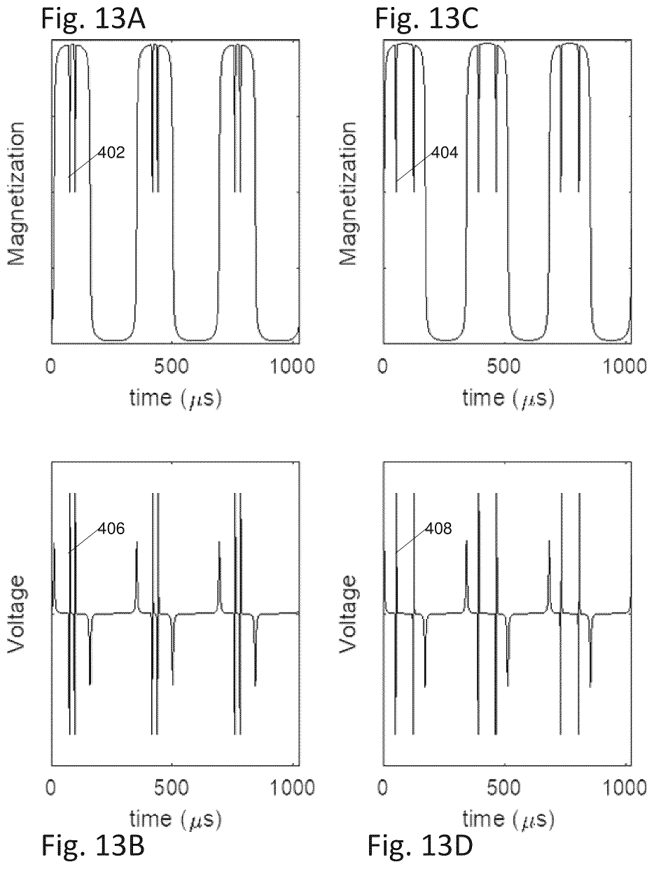

An alternative embodiment of a machine 350 (FIG. 12) to image MNP concentrations uses main magnets 352, 354 that are aligned, not opposed, to create a magnetic field 358 having a magnetic field gradient across an imaging space within which a subject 356, or portion of a subject, may be positioned. A magnetic core 353 couples magnets 352, 354.

Either the main magnets 352, 354 are electromagnets, or scanning electromagnets 357, are provided that can alter intensity of the magnetic field 358 thereby moving a surface (dashed arcs) of resonance from a first location 360 to other locations 362, 364 through a region of interest in the imaging space. At least one pair of RF coils 366 are provided near the region of interest and are driven by an RF generator 368 to excite resonance of MNPs within the surface of resonance or location 360. Sense coils 370 are provided to receive signals from MNPs as they realign with magnetic field 358. EPR RF coils 391 are provided to add a pulsed EPR drive to the MNPs and driven by a pulsed EPR RF driver 393 under control of computer 374, Sense coils are coupled through a lock-in amplifier 372 or other amplifier/analog-to-digital converter to provide sense signals to an imaging computer 374 that controls magnets and RF sources plus analyzes sense signals to determine when resonance disturbs alignment of MNPs with magnetic field 358. Computer 374 also controls RF generator 368 and a scan generator 376 that drives scanning electromagnets 358 or main magnets 352, 354 to move the surface of resonance. In a particular embodiment, computer 374 may also control an audio drive generator 378, coupled to drive coils 380.

Operation of the embodiment of FIG. 12 is similar to that of FIG. 9 in that timing of the signal relative to the audio signal waveform, as analyzed by computer 374, is determined; this timing however provides not a spectrum but surfaces on which the MNPs are located within the subject or biological sample in the region of interest within the imaging space.

Imaging in the system of FIG. 12 results because when magnetic field 358 having a spatial intensity gradient is swept in strength across a subject or biological specimen while a constant RF electromagnetic field is applied, MNPs in the subject or specimen resonate with the constant RF essentially only along a surface where the magnetic field induces a Larmor frequency in the MNPs that matches the RF frequency. For example, if the nanoparticles are located where the magnetic field reaches resonance only late in the sweep, the nanoparticle magnetization is that of FIG. 13A and detectable signals produced by the MNPs are of FIG. 13B. If the MNPs are located where the magnetic field reaches resonance earlier in the sweep, the nanoparticle magnetization is that of FIG. 13C and detectable signals are those of FIG. 13D. The dips 402, 404 in nanoparticle magnetization and peaks 406, 408 in detectable signals are because the resonance disrupts alignment of the MNPs, and they must realign as the resonance passes them.

In the embodiment of FIG. 12, after determining surfaces within the subject or biological sample having resonating MNPs, either the subject or biological sample is rotated relative to the magnetic field and/or the magnetic field is rotated by, for example, deactivating main magnets 352, 354 and activating another set of main magnets (not shown in FIG. 12 for simplicity but similar to those of the MNP hyperthermia treatment machine of FIG. 14) to provide a rotated magnetic field, by using a mechanical rotator 384 about an axis 385 operating under control of computer 374, or both—in a particular embodiment multiple sets of main magnets are provided to provide surfaces of resonance at differing angles from vertical about a horizontal axis 390 through the region of interest while a rotator provides surfaces at different angles of rotation about the axis 385. Additional surfaces are determined within the subject or biological sample where resonating MNPs are located at multiple angles of rotation 386. After multiple surfaces are determined each at a different rotation both about the axis 385 and about a horizontal axis 390, computer 374 analyzes the determined surfaces where resonating MNPs are located and reconstructs locations of the resonating MNPs in a voxel-based three-dimensional model of the subject or biological sample by fitting concentration parameters of the three-dimensional model to signal strengths of observed surfaces in a manner similar to MRI reconstruction.

In an alternative embodiment resembling that of FIG. 12, the bias magnetic field, including the magnetic field gradients, is provided by electromagnets of an MRI machine such as commonly used to image concentrations of unpaired hydrogen protons, although the electromagnets may be operated significantly different—usually lower−currents for EPR imaging of magnetic nanoparticles than for MRI imaging. Since EPR resonance occurs at a frequency that increases with magnetic field strength, and for the same magnetic field strength at far higher frequencies than the hydrogen nuclear magnetic resonances imaged by MRI machines in normal operation, the MRI machine bias field magnets are operated at far lower currents than during MRI operation. By reducing the MRI machine bias field strength, the RF frequency of resonance is low enough to penetrate a subject or biological material containing MNPs and disposed within the MRI machine. In an alternative embodiment, where the MRI machine is a low-field permanent magnet MRI machine configured to image resonance of hydrogen protons in tissue, an additional pair of coils are provided and pulsed with a high current pulse to oppose fields provided by the bias magnets of the MRI machine and reduce field strength sufficiently that the RF frequency is low enough to penetrate a subject or biological material containing MNPs and disposed within the MRI machine.

We note that the EPR resonant frequency of MNPs, as well as the FMR resonant frequency of MNPs depends in part on composition of the MNPs, and that MNPs may be made with iron oxide cores, with a ferrite core, with metallic iron cores, with a cobalt-containing magnetic core, with an iron-platinum alloy core, or with cores of other magnetic alloys. Further, the FMR resonant frequency depends in part on nanoparticle size and viscosity of surrounding fluids. The magnetic cores can be either uniform of a single magnetic material or in “core-shell” or “sandwich” geometries with two magnetic materials. The two materials can be of different permeability allowing one material to flip magnetizations more or less easily.

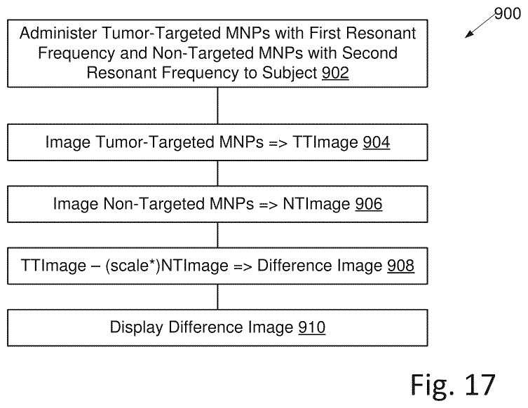

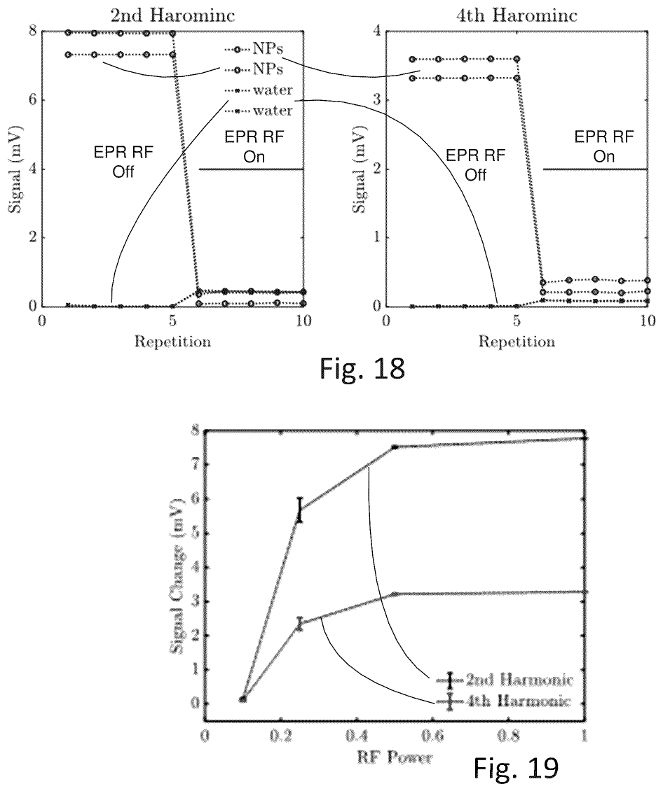

It is therefore possible to form a duplex contrast agent comprising first and second MNPs where the first MNPs are labeled with a first ligand or antibody and the second MNPs are either unlabeled or labeled with a second ligand or antibody. In an embodiment of enhanced MNP imaging 900 (FIG. 17), after administration of this duplex contrast agent either as a single duplex agent or as separate injections to a subject 902 (FIG. 9) biological sample, we image the first MNPs 904 with a first applied EPR frequency to form a first three-dimensional model TTImage of the subject or biological sample and imaging the second MNPs 906 with a second EPR frequency differing from the first EPR frequency (or alternatively with a second base magnetic field strength) to form a second three-dimensional model NTImage of the subject or biological sample. We can display the first and second three-dimensional models separately to a user or can scale as necessary and subtract 908 the second three-dimensional model NTImage from the first three-dimensional model TTImage to form a difference three-dimensional model and display 910 the difference three-dimensional model to the user, display may be done in tomographic slices as known in three dimensional imaging. When first MNPs are targeted to tumor with a first ligand, antibody, or other selective agent so first MNPs concentrate in tumor to a greater extent than in normal tissue, while second MNPs are nontargeted, the difference three-dimensional model cancels background first MNPs and thus increases contrast of tumor over normal tissue.

To review, a method of imaging 800 magnetic nanoparticle concentrations in a subject (FIG. 8) operates by administering MNPs 802 to the subject and allowing them to reach desired organs. A magnetic bias field is then applied 804 to the subject, and pulses of a radio frequency field are applied 806 to the subject at an electron paramagnetic resonant frequency of MNPs in the magnetic bias field forming a surface of resonance within the subject. This then alters magnetization of the MNPs allowing a ferromagnetic resonance to occur at a reasonably low frequency, which can be stimulated by applying an appropriate FMR resonance frequency 807 and observing a response. Either the magnetic bias field strength or the radio frequency is swept 808 through the subject while resonances are observed 810 with appropriate sense coils and electronics. The magnetic bias field is then rotated 812 relative to the subject. Steps of applying pulses of the radio frequency field 806 forming a surface of resonance within the subject, sweeping 808 the magnetic field strength or sweeping the radio frequency while observing resonances 810, and rotating the magnetic field gradient 812 are repeated 814 until sufficient data has been obtained. A computer then sets up a voxel-based model of the subject and uses the observations of resonances to reconstruct MNP concentrations at each voxel within the model. The MNP concentrations are 3D images that may then be displayed 818 as tomographic slices.

In particular embodiments, the MNPs are tagged with antibodies specific for a specific tumor type, and the 3D images represent tumor locations within the subject. In another embodiment, the MNPs are tagged with a ligand capable of binding to a particular tissue type, and the 3D images represent distribution of that tissue type within the subject.

Antibody-tagged MNPs with one type of magnetic properties may be administered to a patient with MNPs having a second type of magnetic properties without the antibody tag. FIG. 17 is a flowchart of a method of imaging both types of MNPs and using image subtraction to increase tumor contrast.

Similarly, heating is accomplished by applying a magnetic field and pulses of electromagnetic radiation at the EPR frequency, which disrupts the MNP magnetization and allows a low-frequency FMR resonance to occur in the MNPs. An RF field at the FMR resonance frequency is applied to heat the MNPs while sparing adjacent tissue.

A significant difference between the present system and many other magnetic nanoparticle imaging systems is that there is no “field free point” in the sample space because presence of a magnetic field at a magnetic nanoparticle is necessary for EPR resonances to occur. Further, for both imaging and heating, the EPR resonance is used to produce a low frequency FMR resonance that can be stimulated or observed with lower frequency electromagnetic radiation.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween. It is also anticipated that steps of methods may be performed in an order different from that illustrated and still be within the meaning of the claims that follow.

What is claimed is:

1. A magnetic nanoparticle (MNP) electron paramagnetic resonance machine comprising:
at least one driver and associated coil producing a low frequency alternating magnetic field (LF-AMF) across a sample space; and
at least one RF driver and RF field coil configured to provide an RF field at an electron paramagnetic resonance (EPR) frequency and a ferromagnetic resonance (FMR) frequency of unpaired electrons in target MNPs in the sample space;
at least one LF-AMF detection module configured to detect magnetization induced by the LF-AMF magnetic field in the sample space; and
a computer configured to control the at least one magnet and RF driver and produce resonance spectra from the target MNPs, images of MNP concentrations from the detected magnetization, or to heat the target MNPs.

2. The MNP electron paramagnetic resonance machine of claim 1 further comprising at least one bias magnet configured to provide a bias magnetic field in the sample space.

3. The MNP electron magnetic resonance machine of claim 1 wherein the LF-AMF detection module comprises a magnetometer or a pickup coil.

4. The MNP electron resonance machine of claim 3 wherein the LF-AMF detection module comprises a balancing coil configured to null signals produced in the LF-AMF detection module in absence of target MNPs in the sample space.

5. The MNP electron magnetic resonance machine of claim 1 further comprising at least one RF pickup coil to record the RF field allowing measurement of the RF absorbed by the target MNPs.

6. The MNP electron magnetic resonance machine of claim 1, configured to heat the target MNPs by controlling absorption of energy using the RF field.

7. The MNP electron magnetic resonance machine of claim 2, configured to detect the MNPs by using pulsed or continuous RF at an EPR/FMR resonant frequency of the target MNPs to alter the magnetization of the target MNPs and sensing the target MNPs at the LF-AMF magnetic field.

8. The MNP electron magnetic resonance machine of claim 1, the computer configured to select and heat the target MNPs by selective absorption of energy from the RF field.

9. The MNP electron magnetic resonance machine of claim 1, configured to improve the detection of the MNPs by altering the magnetization produced by the MNPs using the RF field.

2. MNP electron magnetic resonance machine of claim 2, the at least one bias magnet configured to provide a magnetic field to the sample space and further comprising an MRI RF Driver and MRI RF coils being configured to provide an RF field at resonant frequency of hydrogen protons to make magnetic resonance images of hydrogen in the sample space.

11. An MNP heat-treatment machine comprising the MNP machine of claim 2, wherein at least one RF coil is driven with sufficient power to heat the target MNPs at a frequency that is the FMR or EPR frequency of the MNPs.

12. The MNP heat-treatment machine of claim 11, the computer configured to determine MNP Brownian motion spectra to monitor temperature of the target MNPs during MNP heating.

13. The MNP heat-treatment machine of claim 11, the computer configured to map temperature through the sample space from MNP Brownian motion spectra of the target MNPs.

14. The MNP heat-treatment machine of claim 11, the bias magnetic field having a gradient and the heating of the target MNPs being performed along a surface within the sample space.

15. The MNP heat-treatment machine of claim 14, further comprising apparatus configured to rotate a subject in the sample space relative to the magnetic field.

16. The machine of claim 2, the computer configured to use the FMR to produce the image of the MNP concentrations in the sample space using a magnetic bias field with a gradient.

17. The machine of claim 1, configured to produce resonance spectra.

18. A method of imaging first magnetic nanoparticle (MNP) concentrations in a subject comprising:
applying a magnetic bias field having a gradient to the subject;
applying a first radio frequency field to the subject at an electron paramagnetic resonant (EPR) frequency of the first MNPs in the magnetic bias field;
applying a second radio frequency field to the subject at a ferromagnetic resonance (FMR) frequency of the first MNPs in the magnetic bias field;
sweeping a parameter selected from the group consisting of a strength of the magnetic bias field, the first radio frequency, and the second radio frequency, to sweep a surface of resonance through the subject;
observing electron FMRs of the first MNPs;
rotating the magnetic bias field relative to the subject and sweeping the surface of resonance through the subject while observing additional FMR resonances of the first MNPs; and
reconstructing first MNP concentrations in a first three-dimensional model of the subject.

19. The method of claim 18 further comprising imaging second magnetic nanoparticle (MNP) concentrations in the subject by a method comprising:
applying the magnetic bias field having a gradient to the subject;
applying a third radio frequency field to the subject at an FMR frequency of the second MNPs in the magnetic bias field;
applying a fourth radio frequency field to the subject at a ferromagnetic resonance (FMR) frequency of the second MNPs;
sweeping a parameter selected from the group consisting of the strength of the magnetic bias field, the third radio frequency, and the fourth radio frequency to sweep a surface of resonance through the subject;
observing FMRs of the second MNPs;
rotating the magnetic bias field relative to the subject and sweeping the surface of resonance through the subject while observing additional FMRs of the second MNPs; and
reconstructing second MNP concentrations in a second three-dimensional model of the subject.

20. The method of claim 19 further comprising subtracting the second three-dimensional model of the subject from the first three-dimensional model of the subject.

21. The method of claim 18 wherein the MNPs are complexed with antibodies to a particular tissue type.

22. The method of claim 18 further comprising heating the MNPs by applying radio frequency energy at a frequency of electron paramagnetic resonances of the MNPs.

23. The method of claim 22 further comprising observing an MNP Brownian motion spectrum to determine a temperature of the MNPs.

24. The method of claim 22 further comprising heating the MNPs by applying radio frequency energy at a frequency of electron paramagnetic resonances of the MNPs.

25. The method of claim 24 further comprising observing an MNP Brownian motion spectrum to map a temperature of the MNPs within the subject.
